SEQUENCE LISTING (68 sequences)

SEQ ID NO: 1 - protein, 118 aa, synthetic construct
  REGION 1..118: note = SiRP alpha variant
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKP

SEQ ID NO: 2 - protein, 118 aa, synthetic construct
  REGION 1..118: note = human wild type SIRP alpha
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKP

SEQ ID NO: 3 - protein, 119 aa, synthetic construct
  REGION 1..119: note = wild type SIRP gamma
EEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPS

SEQ ID NO: 4 - protein, 118 aa, synthetic construct
  REGION 1..118: note = SIRP gamma variant 1 (V1)
EEELQIIQPEKLLLVTVGKTATLHCTITSLFPVGPVLWFRGVGPGRVLIYNQRQGPFPRVTTVSDTTKRNNMDFSIRISSITPADVGTYYCIKFRKGSPENVEFKSGPGTEMALGAKP

SEQ ID NO: 5 - protein, 118 aa, synthetic construct
  REGION 1..118: note = SIRP gamma variant 2 (V2)
EEELQIIQPEKLLLVTVGKTATLHCTVTSLFPVGPVLWFRGVGPGRVLIYNQRQGPFPRVTTVSDTTKRNNMDFSIRISSITPADVGTYYCIKFRKGSPENVEFKSGPGTEMALGAKP

SEQ ID NO: 6 - protein, 117 aa, synthetic construct
  REGION 1..117: note = SiRP alpha variant
EEEVQIIQPDKSVSVAAGESAILHCTITSLFPVGPIQWFRGAGPARVLIYNQRQGPFPRVTTISETTRRENMDFSISISNITPADAGTYYCIKFRKGSPDTEFKSGAGTELSVRAKP

SEQ ID NO: 7 - protein, 117 aa, synthetic construct
  REGION 1..117: note = SiRP alpha D1 domain
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKP

SEQ ID NO: 8 - protein, 118 aa, synthetic construct
  REGION 1..118: note = SiRP alpha variant
EEEVQIIQPDKSVSVAAGESAILHCTITSLFPVGPIQWFRGAGPARVLIYNQRQGPFPRVTTISETTRRENMDFSISISNITPADAGTYYCIKFRKGSPDTEFKSGAGTELSVRAKPS

SEQ ID NO: 9 - protein, 119 aa, synthetic construct
  REGION 1..119: note = SiRP alpha variant
  VARIANT 1: note = unknown amino acid
XLLFNKTKSVEFTFGNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSTR

SEQ ID NO: 10 - protein, 118 aa, synthetic construct; identical to SEQ ID NO: 8
  REGION 1..118: note = SiRP alpha variant

SEQ ID NO: 11 - protein, 121 aa, synthetic construct
  REGION 1..121: note = SiRP alpha variant
  VARIANT 1: note = unknown amino acid
XLLFNKTKSVEFTFGNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWSTR

SEQ ID NO: 12 - protein, 120 aa, synthetic construct
  REGION 1..120: note = SiRP alpha variant
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSTR

SEQ ID NO: 13 - protein, 120 aa, synthetic construct; identical to SEQ ID NO: 12
  REGION 1..120: note = SiRP alpha variant

SEQ ID NO: 14 - protein, 118 aa, synthetic construct
  REGION 1..118: note = SiRP alpha variant
EEELQVIQPDKSVSVAAGESAILHCTITSLIPVGPIQWFRGAGPARELIYNQREGHFPRVTTVSETTRRENMDFSISISNITPADAGTYYCVKFRKGSPDTEVKSGAGTELSVRAKPS

SEQ ID NO: 15 - protein, 118 aa, synthetic construct
  REGION 1..118: note = SiRP alpha variant
EEEVQVIQPDKSVSVAAGESAILHCTLTSLIPVGPIQWFRGAGPARVLIYNQRQGHFPRVTTVSEGTRRENMDFSISISNITPADAGTYYCIKFRKGSPDTEFKSGAGTELSVRAKPS

SEQ ID NO: 16 - protein, 118 aa, synthetic construct
  REGION 1..118: note = SiRP alpha variant
EEEVQIIQPDKSVSVAAGESVILHCTITSLTPVGPIQWFRGAGPARLLIYNQREGPFPRVTTVSETTRRENMDFSISISNITPADAGTYYCVKLRKGSPDTEFKSGAGTELSVRAKPS

SEQ ID NO: 17 - protein, 118 aa, synthetic construct
  REGION 1..118: note = SiRP alpha variant
EEELQIIQPDKSVSVAAGESAILHCTITSLSPVGPIQWFRGAGPARVLIYNQRQGPFPRVTTVSEGTKRENMDFSISISNITPADAGTYYCIKLRKGSPDTEFKSGAGTELSVRAKPS

SEQ ID NO: 18 - protein, 118 aa, synthetic construct
  REGION 1..118: note = SiRP alpha variant
EEEIQVIQPDKSVSVAAGESVIIHCTVTSLFPVGPIQWFRGAGPARVLIYNQRQGRFPRVTTVSEGTKRENMDFSISISNITPADAGTYYCVKVRKGSPDTEVKSGAGTELSVRAKPS

SEQ ID NO: 19 - protein, 118 aa, synthetic construct
  REGION 1..118: note = SiRP alpha variant
EEEVQIIQPDKSVSVAAGESIILHCTVTSLFPVGPIQWFRGAGPARVLIYNQREGRFPRVTTVSEGTRRENMDFSISISNITPADAGTYYCIKLRKGSPDTEFKSGAGTELSVRAKPS

SEQ ID NO: 20 - protein, 118 aa, synthetic construct
  REGION 1..118: note = SiRP alpha variant
EEEVQLIQPDKSVSVAAGESAILHCTVTSLFPVGPIQWFRGAGPARVLIYNQREGPFPRVTTVSEGTKRENMDFSISISNITPADAGTYYCIKFRKGSPDTEVKSGAGTELSVRAKPS

SEQ ID NO: 21 - protein, 119 aa, synthetic construct
  REGION 1..119: note = SiRP alpha variant
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPS

SEQ ID NO: 22 - protein, 118 aa, synthetic construct
  REGION 1..118: note = SiRP alpha variant
EEELQIIQPDKSVSVAAGESAILHCTITSLFPVGPIQWFRGAGPARLLIYNQRQGPFPRVTTVSETTKRENMDFSISISNITPADAGTYYCVKFRKGSPDTEFKSGAGTELSVRAKPS

SEQ ID NO: 23 - protein, 118 aa, synthetic construct
  REGION 1..118: note = SiRP alpha variant
EEEVQIIQPDKSVSVAAGESAILHCTITSLFPVGPIQWFRGAGPARVLIYNQKQGPFPRVTTISETTRRENMDFSISISNITPADAGTYYCIKFRKGSPDTEFKSGAGTELSVRAKPS

SEQ ID NO: 24 - protein, 118 aa, synthetic construct
  REGION 1..118: note = SiRP alpha variant
EEELQIIQPDKSVSVAAGESAILHCTITSLTPVGPIQWFRGAGPARVLIYNQRQGPFPRVTTVSEGTRRENMDFSISISNITPADAGTYYCIKFRKGSPDTEVKSGAGTELSVRAKPS

SEQ ID NO: 25 - protein, 119 aa, synthetic construct
  REGION 1..119: note = SiRP alpha variant
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPS

SEQ ID NO: 26 - protein, 118 aa, synthetic construct
  REGION 1..118: note = SiRP alpha variant
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 27 - protein, 118 aa, synthetic construct
  REGION 1..118: note = SiRP alpha variant
EEELQVIQPDKSVSVAAGESAILLCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 28 - protein, 119 aa, synthetic construct
  REGION 1..119: note = SiRP alpha variant
EEGLQVIQPDKSVSVAAGESAILHCTATSLIPVGPIQWFRGAGPGRELIYNQKEGHFPRVTTVSDLTKRNNMDFSIRIGNITPADAGTYYCVKFRKGSPDDVEFKSGAGTELSVRAKPS

SEQ ID NO: 29 - protein, 119 aa, synthetic construct
  REGION 1..119: note = SiRP alpha variant
EEELQVIQPDKFVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPS

SEQ ID NO: 30 - protein, 119 aa, synthetic construct
  REGION 1..119: note = SiRP alpha variant
EEELQVIQPDKSVLVAAGETATLRCTATSLIPVGPIQWFRGAGPGRELIYNQKEGHFPRVTTVSDLTKRNNMDFPIRIGNITPADAGTYYCVKFRKGSPDDVEFKSGAGTELSVRAKPS

SEQ ID NO: 31 - protein, 118 aa, synthetic construct
  REGION 1..118: note = SiRP alpha variant
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRGKPS

SEQ ID NO: 32 - protein, 118 aa, synthetic construct
  REGION 1..118: note = SiRP alpha variant
EEELQVIQPDKSVLVAAGETATLRCTATSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 33 - protein, 119 aa, synthetic construct
  REGION 1..119: note = SiRP alpha variant
EEELQVIQPDKSVLVAAGETATLRCTATSLIPVGPIQWFRGAGPGRELIYNQKEGHFPRVTTVSDLTKRNNMDFSIRISNITPADAGTYYCVKFRKGSPDDVEFKSGAGTELSVRAKPS

SEQ ID NO: 34 - protein, 118 aa, synthetic construct; identical to SEQ ID NO: 26
  REGION 1..118: note = SiRP alpha variant

SEQ ID NO: 35 - protein, 119 aa, synthetic construct
  REGION 1..119: note = SiRP alpha variant
  VARIANT 4: note = unknown amino acid
  VARIANT 6: note = unknown amino acid
  VARIANT 21: note = unknown amino acid
  VARIANT 27: note = unknown amino acid
  VARIANT 31: note = unknown amino acid
  VARIANT 47: note = unknown amino acid
  VARIANT 53..54: note = unknown amino acid
  VARIANT 56: note = unknown amino acid
  VARIANT 66: note = unknown amino acid
  VARIANT 68: note = unknown amino acid
  VARIANT 80: note = unknown amino acid
  VARIANT 83: note = unknown amino acid
  VARIANT 92: note = unknown amino acid
  VARIANT 94: note = unknown amino acid
  VARIANT 104: note = unknown amino acid
EEEXQXIQPDKSVLVAAGETXTLRCTXTSLXPVGPIQWFRGAGPGRXLIYNQXXGXFPRVTTVSDXTXRNNMDFSIRIGXITXADAGTYYCXKXRKGSPDDVEXKSGAGTELSVRAKPS

SEQ ID NO: 36 - protein, 118 aa, synthetic construct
  REGION 1..118: note = SiRP alpha variant
  VARIANT 4: note = unknown amino acid
  VARIANT 6: note = unknown amino acid
  VARIANT 21: note = unknown amino acid
  VARIANT 27: note = unknown amino acid
  VARIANT 31: note = unknown amino acid
  VARIANT 47: note = unknown amino acid
  VARIANT 53..54: note = unknown amino acid
  VARIANT 56: note = unknown amino acid
  VARIANT 66: note = unknown amino acid
  VARIANT 68: note = unknown amino acid
  VARIANT 80: note = unknown amino acid
  VARIANT 83: note = unknown amino acid
  VARIANT 92: note = unknown amino acid
  VARIANT 94: note = unknown amino acid
  VARIANT 103: note = unknown amino acid
EEEXQXIQPDKSVSVAAGESXILHCTXTSLXPVGPIQWFRGAGPARXLIYNQXXGXFPRVTTVSEXTXRENMDFSISISXITXADAGTYYCXKXRKGSPDTEXKSGAGTELSVRAKPS

SEQ ID NO: 37 - protein, 118 aa, synthetic construct
  REGION 1..118: note = SiRP alpha variant
  VARIANT 4: note = unknown amino acid
  VARIANT 6: note = unknown amino acid
  VARIANT 21: note = unknown amino acid
  VARIANT 27: note = unknown amino acid
  VARIANT 31: note = unknown amino acid
  VARIANT 47: note = unknown amino acid
  VARIANT 53..54: note = unknown amino acid
  VARIANT 56: note = unknown amino acid
  VARIANT 66: note = unknown amino acid
  VARIANT 68: note = unknown amino acid
  VARIANT 80: note = unknown amino acid
  VARIANT 83: note = unknown amino acid
  VARIANT 92: note = unknown amino acid
  VARIANT 94: note = unknown amino acid
  VARIANT 103: note = unknown amino acid
EEEXQXIQPDKSVSVAAGESXILLCTXTSLXPVGPIQWFRGAGPARXLIYNQXXGXFPRVTTVSEXTXRENMDFSISISXITXADAGTYYCXKXRKGSPDTEXKSGAGTELSVRAKPS

SEQ ID NO: 38 - protein, 119 aa, synthetic construct
  REGION 1..119: note = SiRP alpha variant
  VARIANT 4: note = unknown amino acid
  VARIANT 6: note = unknown amino acid
  VARIANT 21: note = unknown amino acid
  VARIANT 27: note = unknown amino acid
  VARIANT 31: note = unknown amino acid
  VARIANT 47: note = unknown amino acid
  VARIANT 53..54: note = unknown amino acid
  VARIANT 56: note = unknown amino acid
  VARIANT 66: note = unknown amino acid
  VARIANT 68: note = unknown amino acid
  VARIANT 80: note = unknown amino acid
  VARIANT 83: note = unknown amino acid
  VARIANT 92: note = unknown amino acid
  VARIANT 94: note = unknown amino acid
  VARIANT 104: note = unknown amino acid
EEGXQXIQPDKSVSVAAGESXILHCTXTSLXPVGPIQWFRGAGPGRXLIYNQXXGXFPRVTTVSDXTXRNNMDFSIRIGXITXADAGTYYCXKXRKGSPDDVEXKSGAGTELSVRAKPS

SEQ ID NO: 39 - protein, 119 aa, synthetic construct
  REGION 1..119: note = SiRP alpha variant
  VARIANT 4: note = unknown amino acid
  VARIANT 6: note = unknown amino acid
  VARIANT 21: note = unknown amino acid
  VARIANT 27: note = unknown amino acid
  VARIANT 31: note = unknown amino acid
  VARIANT 47: note = unknown amino acid
  VARIANT 53..54: note = unknown amino acid
  VARIANT 56: note = unknown amino acid
  VARIANT 66: note = unknown amino acid
  VARIANT 68: note = unknown amino acid
  VARIANT 80: note = unknown amino acid
  VARIANT 83: note = unknown amino acid
  VARIANT 92: note = unknown amino acid
  VARIANT 94: note = unknown amino acid
  VARIANT 104: note = unknown amino acid
EEEXQXIQPDKFVLVAAGETXTLRCTXTSLXPVGPIQWFRGAGPGRXLIYNQXXGXFPRVTTVSDXTXRNNMDFSIRIGXITXADAGTYYCXKXRKGSPDDVEXKSGAGTELSVRAKPS

SEQ ID NO: 40 - protein, 119 aa, synthetic construct
  REGION 1..119: note = SiRP alpha variant
  VARIANT 4: note = unknown amino acid
  VARIANT 6: note = unknown amino acid
  VARIANT 21: note = unknown amino acid
  VARIANT 27: note = unknown amino acid
  VARIANT 31: note = unknown amino acid
  VARIANT 47: note = unknown amino acid
  VARIANT 53..54: note = unknown amino acid
  VARIANT 56: note = unknown amino acid
  VARIANT 66: note = unknown amino acid
  VARIANT 68: note = unknown amino acid
  VARIANT 80: note = unknown amino acid
  VARIANT 83: note = unknown amino acid
  VARIANT 92: note = unknown amino acid
  VARIANT 94: note = unknown amino acid
  VARIANT 104: note = unknown amino acid
EEEXQXIQPDKSVLVAAGETXTLRCTXTSLXPVGPIQWFRGAGPGRXLIYNQXXGXFPRVTTVSDXTXRNNMDFPIRIGXITXADAGTYYCXKXRKGSPDDVEXKSGAGTELSVRAKPS

SEQ ID NO: 41 - protein, 118 aa, synthetic construct
  REGION 1..118: note = SiRP alpha variant
  VARIANT 4: note = unknown amino acid
  VARIANT 6: note = unknown amino acid
  VARIANT 21: note = unknown amino acid
  VARIANT 27: note = unknown amino acid
  VARIANT 31: note = unknown amino acid
  VARIANT 47: note = unknown amino acid
  VARIANT 53..54: note = unknown amino acid
  VARIANT 56: note = unknown amino acid
  VARIANT 66: note = unknown amino acid
  VARIANT 68: note = unknown amino acid
  VARIANT 80: note = unknown amino acid
  VARIANT 83: note = unknown amino acid
  VARIANT 92: note = unknown amino acid
  VARIANT 94: note = unknown amino acid
  VARIANT 103: note = unknown amino acid
EEEXQXIQPDKSVSVAAGESXILHCTXTSLXPVGPIQWFRGAGPARXLIYNQXXGXFPRVTTVSEXTXRENMDFSISISXITXADAGTYYCXKXRKGSPDTEXKSGAGTELSVRGKPS

SEQ ID NO: 42 - protein, 118 aa, synthetic construct
  REGION 1..118: note = SiRP alpha variant
  VARIANT 4: note = unknown amino acid
  VARIANT 6: note = unknown amino acid
  VARIANT 21: note = unknown amino acid
  VARIANT 27: note = unknown amino acid
  VARIANT 31: note = unknown amino acid
  VARIANT 47: note = unknown amino acid
  VARIANT 53..54: note = unknown amino acid
  VARIANT 56: note = unknown amino acid
  VARIANT 66: note = unknown amino acid
  VARIANT 68: note = unknown amino acid
  VARIANT 80: note = unknown amino acid
  VARIANT 83: note = unknown amino acid
  VARIANT 92: note = unknown amino acid
  VARIANT 94: note = unknown amino acid
  VARIANT 103: note = unknown amino acid
EEEXQXIQPDKSVLVAAGETXTLRCTXTSLXPVGPIQWFRGAGPARXLIYNQXXGXFPRVTTVSEXTXRENMDFSISISXITXADAGTYYCXKXRKGSPDTEXKSGAGTELSVRAKPS

SEQ ID NO: 43 - protein, 119 aa, synthetic construct
  REGION 1..119: note = SiRP alpha variant
  VARIANT 4: note = unknown amino acid
  VARIANT 6: note = unknown amino acid
  VARIANT 21: note = unknown amino acid
  VARIANT 27: note = unknown amino acid
  VARIANT 31: note = unknown amino acid
  VARIANT 47: note = unknown amino acid
  VARIANT 53..54: note = unknown amino acid
  VARIANT 56: note = unknown amino acid
  VARIANT 66: note = unknown amino acid
  VARIANT 68: note = unknown amino acid
  VARIANT 80: note = unknown amino acid
  VARIANT 83: note = unknown amino acid
  VARIANT 92: note = unknown amino acid
  VARIANT 94: note = unknown amino acid
  VARIANT 104: note = unknown amino acid
EEEXQXIQPDKSVLVAAGETXTLRCTXTSLXPVGPIQWFRGAGPGRXLIYNQXXGXFPRVTTVSDXTXRNNMDFSIRISXITXADAGTYYCXKXRKGSPDDVEXKSGAGTELSVRAKPS

SEQ ID NO: 44 - protein, 118 aa, synthetic construct; identical to SEQ ID NO: 36
  REGION 1..118: note = SiRP alpha variant
  VARIANT 4: note = unknown amino acid
  VARIANT 6: note = unknown amino acid
  VARIANT 21: note = unknown amino acid
  VARIANT 27: note = unknown amino acid
  VARIANT 31: note = unknown amino acid
  VARIANT 47: note = unknown amino acid
  VARIANT 53..54: note = unknown amino acid
  VARIANT 56: note = unknown amino acid
  VARIANT 66: note = unknown amino acid
  VARIANT 68: note = unknown amino acid
  VARIANT 80: note = unknown amino acid
  VARIANT 83: note = unknown amino acid
  VARIANT 92: note = unknown amino acid
  VARIANT 94: note = unknown amino acid
  VARIANT 103: note = unknown amino acid

SEQ ID NO: 45 - protein, 119 aa, synthetic construct
  REGION 1..119: note = SiRP alpha variant
  VARIANT 3..4: note = unknown amino acid
  VARIANT 6: note = unknown amino acid
  VARIANT 12: note = unknown amino acid
  VARIANT 14: note = unknown amino acid
  VARIANT 20..22: note = unknown amino acid
  VARIANT 24: note = unknown amino acid
  VARIANT 27: note = unknown amino acid
  VARIANT 31: note = unknown amino acid
  VARIANT 45: note = unknown amino acid
  VARIANT 47: note = unknown amino acid
  VARIANT 53..54: note = unknown amino acid
  VARIANT 56: note = unknown amino acid
  VARIANT 65..66: note = unknown amino acid
  VARIANT 68: note = unknown amino acid
  VARIANT 70: note = unknown amino acid
  VARIANT 75: note = unknown amino acid
  VARIANT 77: note = unknown amino acid
  VARIANT 79..80: note = unknown amino acid
  VARIANT 83: note = unknown amino acid
  VARIANT 92: note = unknown amino acid
  VARIANT 94: note = unknown amino acid
  VARIANT 101..102: note = unknown amino acid
  VARIANT 104: note = unknown amino acid
  VARIANT 116: note = unknown amino acid
EEXXQXIQPDKXVXVAAGEXXXLXCTXTSLXPVGPIQWFRGAGPXRXLIYNQXXGXFPRVTTVSXXTXRXNMDFXIXIXXITXADAGTYYCXKXRKGSPDXXEXKSGAGTELSVRXKPS

SEQ ID NO: 46 - protein, 118 aa, synthetic construct; identical to SEQ ID NO: 14
  REGION 1..118: note = SiRP alpha variant

SEQ ID NO: 47 - protein, 118 aa, synthetic construct; identical to SEQ ID NO: 15
  REGION 1..118: note = SiRP alpha variant

SEQ ID NO: 48 - protein, 118 aa, synthetic construct; identical to SEQ ID NO: 16
  REGION 1..118: note = SiRP alpha variant

SEQ ID NO: 49 - protein, 118 aa, synthetic construct; identical to SEQ ID NO: 17
  REGION 1..118: note = SiRP alpha variant

SEQ ID NO: 50 - protein, 118 aa, synthetic construct; identical to SEQ ID NO: 18
  REGION 1..118: note = SiRP alpha variant

SEQ ID NO: 51 - protein, 118 aa, synthetic construct; identical to SEQ ID NO: 19
  REGION 1..118: note = SiRP alpha variant

SEQ ID NO: 52 - protein, 118 aa, synthetic construct; identical to SEQ ID NO: 20
  REGION 1..118: note = SiRP alpha variant

SEQ ID NO: 53 - protein, 119 aa, synthetic construct; identical to SEQ ID NO: 21
  REGION 1..119: note = SiRP alpha variant

SEQ ID NO: 54 - protein, 118 aa, synthetic construct; identical to SEQ ID NO: 22
  REGION 1..118: note = SiRP alpha variant

SEQ ID NO: 55 - protein, 118 aa, synthetic construct; identical to SEQ ID NO: 23
  REGION 1..118: note = SiRP alpha variant

SEQ ID NO: 56 - protein, 118 aa, synthetic construct; identical to SEQ ID NO: 24
  REGION 1..118: note = SiRP alpha variant

SEQ ID NO: 57 - protein, 474 aa, synthetic construct
  REGION 1..474: note = human wild type SIRP-alpha(h-wa) full sequence
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 58 - protein, 474 aa, synthetic construct
  REGION 1..474: note = variant SIRP-alpha(va) full sequence
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 59 - protein, 359 aa, synthetic construct
  REGION 1..359: note = wild type SIRP-gamma(wg) full sequence
EEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVTVHQKDQSSDATPGPASSLTALLLIAVLLGPIYVPWKQKT

SEQ ID NO: 60 - protein, 358 aa, synthetic construct
  REGION 1..358: note = variant SIRP-gamma-1 (vg-1) full sequence
EEELQIIQPEKLLLVTVGKTATLHCTITSLFPVGPVLWFRGVGPGRVLIYNQRQGPFPRVTTVSDTTKRNNMDFSIRISSITPADVGTYYCIKFRKGSPENVEFKSGPGTEMALGAKPAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVTVHQKDQSSDATPGPASSLTALLLIAVLLGPIYVPWKQKT

SEQ ID NO: 61 - protein, 358 aa, synthetic construct
  REGION 1..358: note = variant SIRP-gamma-2 (vg-2) full sequence
EEELQIIQPEKLLLVTVGKTATLHCTVTSLFPVGPVLWFRGVGPGRVLIYNQRQGPFPRVTTVSDTTKRNNMDFSIRISSITPADVGTYYCIKFRKGSPENVEFKSGPGTEMALGAKPAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVTVHQKDQSSDATPGPASSLTALLLIAVLLGPIYVPWKQKT

SEQ ID NO: 62 - DNA, 357 bp, synthetic construct
  misc_feature 1..357: note = polynucleotide encoding SiRP alpha variant
  source 1..357: mol_type = other DNA
gaagaggagctgcagatcatccagcctgacaagtccgtgctggtcgctgctggtgaaactgccactctgcgttgtacgattaccagcctgttcccggtgggtccaatccagtggttccgtggtgctggtccgggtcgtgttctgatctacaaccagcgtcaaggtccgttcccgcgtgtaactaccgttagcgataccacgaagcgtaacaacatggacttttccatccgcattggcaatattaccccggccgacgcgggcacctactattgcatcaaatttcgcaaaggctccccggatgatgtagaatttaaatctggcgcaggcaccgaactgtctgttcgcgcaaaaccgtaa

SEQ ID NO: 63 - DNA, 357 bp, synthetic construct
  misc_feature 1..357: note = human wild type SIRP alpha
  source 1..357: mol_type = other DNA
gaggaggaattacaggtcattcaaccagataaatcggtcttagtagcagccggagagacagctacattgagatgtacggcgacaagccttattcccgtggggccgatccaatggtttcgcggggcaggccccggaagagaattgatttacaaccagaaggagggtcatttccctcgcgtgacgacggtcagcgacttaactaagcgtaataacatggatttttcaataagaataggcaatataactccggccgacgcagggacgtactactgtgttaaattccggaagggatctccggatgatgtcgagttcaaatctggggcgggtacagaattgagcgttcgggcaaagccctaa

SEQ ID NO: 64 - DNA, 354 bp, synthetic construct
  misc_feature 1..354: note = wild type SIRP gamma
  source 1..354: mol_type = other DNA
gaggaagaattgcaaatgatccagccggaaaaattattactggttaccgtgggaaaaacggcgacccttcattgcacagtcacgtccctgttgccggtaggtccagttttgtggttccggggggttggaccagggcgtgaactgatctataatcaaaaggaaggtcattttccgcgcgtgaccacagtgagcgatttgactaaacggaacaatatggacttctcgatccgcatttctagtattacaccggcggacgttggcacttattattgcgtcaagttccgcaaaggaagtcctgagaacgtagagttcaagtccggtcctggcactgagatggctttgggtgctaaaccc

SEQ ID NO: 65 - DNA, 354 bp, synthetic construct
  misc_feature 1..354: note = polynucleotide encoding SIRP gamma variant 1
  source 1..354: mol_type = other DNA
gaagaggagctgcagatcatccagcctgacaagtccgtgctggtcgctgctggtgaaactgccactctgcgttgtacgattaccagcctgttcccggtgggtccaatccagtggttccgtggtgctggtccgggtcgtgttctgatctacaaccagcgtcaaggtccgttcccgcgtgtaactaccgttagcgataccacgaagcgtaacaacatggacttttccatccgcattggcaatattaccccggccgacgcgggcacctactattgcatcaaatttcgcaaaggctccccggatgatgtagaatttaaatctggcgcaggcaccgaactgtctgttcgcgcaaaaccg

SEQ ID NO: 66 - DNA, 354 bp, synthetic construct
  misc_feature 1..354: note = polynucleotide encoding SIRP gamma variant 2
  source 1..354: mol_type = other DNA
gaagaggaattacaaatcatacaacctgaaaagctgttattggtcaccgtaggcaaaaccgctactctgcactgcactgtgacgtccctttttcctgttggtcctgtcttatggtttcgtggagtcggtccgggtcgggttcttatctataaccagcggcaaggaccattcccacgggttaccacggtttcggacacaacgaaacgcaataacatggatttttccattcggatttcaagcatcactccggccgacgttggaacttattactgcataaagtttagaaagggatctccggagaacgtagaatttaagtctggtccaggtactgagatggcccttggagcgaagccg

SEQ ID NO: 67 - DNA, 24 bp, synthetic construct
  misc_feature 1..24: note = Forward primer
  source 1..24: mol_type = other DNA
aaacatatggaagaggagctgcag

SEQ ID NO: 68 - DNA, 27 bp, synthetic construct
  misc_feature 1..27: note = Reverse primer
  source 1..27: mol_type = other DNA
aaaaagctttcaattcagatcctcttc

(19) United States
(12) Patent Application Publication
(10) Pub. No.: US 2024/0209063 A1
(43) Pub. Date: Jun. 27, 2024

(54) NOVEL EXOSOME-BASED ANTICANCER AGENT

(71) Applicant: SHIFTBIO INC., Seoul (KR)

(72) Inventors: Eun-ee KOH, Seoul (KR); Eun Jung LEE, Seoul (KR); Yoo Soo YANG, Seoul (KR); In-San KIM, Seoul (KR)

(21) Appl. No.: 18/596,778

(22) Filed: Mar. 6, 2024

Related U.S. Application Data
(63) Continuation of application No. 17/712,155, filed on Apr. 3, 2022, now Pat. No. 11,952,412.
Application No. 17/712,155 is a continuation of application No. 16/475,052, filed on Jun. 28, 2019, now Pat. No. 11,319,360.

(30) Foreign Application Priority Data
Dec. 29, 2016 (KR) 10-2016-0182573

(51) Int. Cl. C07K 14/71; A61K 38/00; A61P 35/00; C12N 9/12

(52) U.S. Cl. CPC C07K 14/71; A61P 35/00; C12N 9/12; C12Y 207/10001; A61K 38/00; C07K 2319/03

(57) ABSTRACT

The present invention provides a recombinant exosome and uses thereof. More particularly, the present invention provides a recombinant exosome wherein a phagocytosis promoting protein is presented on the surface of the exosome.

### CROSS-REFERENCE TO RELATED APPLICATIONS

This application is a continuation application of U.S. Application Ser. No. 17/712,155 filed on Apr. 3, 2022, which is a continuation application of U.S. application Ser. No. 16/475,052 filed on Jun. 28, 2019, which is the U.S. national stage of International Application No. PCT/KR2017/015784 filed on Dec. 29, 2017, which claims priority to Korean Patent Application No. 10-2016-0182573 filed on Dec. 29, 2016, the contents of which applications are incorporated by reference in their entirety.

### REFERENCE TO ELECTRONIC SEQUENCE LISTING

The application contains a Sequence Listing which has been submitted electronically in .XML format and is hereby incorporated by reference in its entirety. Said .XML copy, created on Mar. 6, 2024, is named “Seq_SIW-P30001C2-US.xml” and is 162,063 bytes in size. The sequence listing contained in this .XML file is part of the specification and is hereby incorporated by reference herein in its entirety.

### TECHNICAL FIELD

The present invention relates to a novel anticancer agent, and more specifically, to a novel anticancer agent that increases the phagocytosis of cancer cells.

### BACKGROUND

Tumors avoid immune surveillance by expressing activating and inhibiting ligands that interact with receptors existing on the surface of immune cells to survive and proliferate. This interaction between tumor and immune cells prevents the tumor from being killed by the immune system (Pardoll, D. M., Nat. Rev. Cancer. 12: 252-264, 2012). One of mechanisms of immunological escape of tumor is the overexpression of CD47 which allows tumors to avoid innate immune surveillance. When CD47 binds to signal-regulatory protein a (SIRPa) of innate immune cells such as macrophages, the innate immune cells activate “do not eat me” signaling which leads to the avoidance of phagocytosis of the tumor by themselves, suggesting strong evidence for the therapeutic targeting of the CD47-SIRPa interaction since abundant CD47 expression in various malignant tumor cells results in a lower survival rate in cancer patients. Since the N-terminus of SIRPα contains the immunoglobulin superfamily V-like domain that interacts with the N-terminus of CD47, several competitive antagonists have been developed to block the interaction. Human CD47 blocking monoclonal antibody (CD47 mAb) proved efficacy in a variety of preclinical tumor models and triggered T cell mediated immunogenic destruction of tumors (Tseng, D. et al., Proc. Natl. Acad. Sci. USA 110: 11103-11108, 2013). In addition, CD47-SIRPα interactions may exhibit antagonistic effects with recombinant SIRPα proteins or SIRPα-FC fusion proteins. However, since weak interactions between native CD47 and SIRPα may limit the usefulness of wild-type SIRPα proteins as therapeutic antagonists, high-affinity mutants of SIRPα were developed and antagonized CD47 in cancer cells successfully. However, SIRPα mutants only acted as adjuvants to tumor-specific antibodies, but they did not stimulate phagocytosis and inhibition of tumor growth beyond expectations (Sockolosky, J. T. et al., Proc. Natl. Acad. Sci. USA 113: E2646-E2654, 2016). In this regard, US Patent Application Publication No. 2015-0376288 discloses a therapeutic method for treating a pathogen infection by administering an agent that reduces the binding of CD47 to SIRPα in an infected cell on a host phagocytic cell.

### SUMMARY

However, in the case of the above-mentioned prior art, administration of an anti-CD47 agent for treatment of infectious diseases reduces binding of SIRPα, which is unsuitable as a therapeutic agent for chemotherapy.

Accordingly, the present invention has been made to solve the above-mentioned problems, and it is an object of the present invention to provide a novel exosome-based anticancer agent which is effective for chemotherapy for removing cancer cells by increasing phagocytosis of the cancer cell by macrophages and dendritic cells. However, these problems are exemplary and do not limit the scope of the present invention.

In an aspect of the present invention, there is provided a recombinant exosome in which a phagocytosis promoting protein is presented on the surface thereof.

In another aspect of the present invention, there is provided a pharmaceutical composition for anticancer therapy comprising a therapeutically effective amount of the recombinant exosome and a pharmaceutically acceptable carrier.

In another aspect of the present invention, there is provided a method of treating cancer in a subject comprising administering therapeutically effective amount of the exosome of claim 1 to the subject.

According to one embodiment of the present invention as described above, the production of effective exosome-based anticancer agent whose phagocytic activity of macrophages and dendritic cells against cancer cells is improved can be accomplished. In particular, the recombinant exosome in which SIRP is present on the surface thereof according to an embodiment of the present invention can stimulated the anti-cancer immune response more effectively by blocking SIRP-CD47 interaction with only small amount of exosomes since the SIRP is clustered in the form of a lipid raft on the surface of the exosome so that has high binding activity to the CD47 protein which is also clustered in the form of a lipid raft on the cancer cell surface. However, the scope of the present invention is not limited by these effects.

### BRIEF DESCRIPTION OF THE DRAWINGS

FIG. 1 is a schematic diagram representing the structure of a plasmid DNA expressing a SIRPα mutant (SIRPα-exosomes) according to an embodiment of the present invention.

FIG. 2 shows a Western blot gel photograph showing the expression of recombinant exosome (SIRPα-exosomes) prepared according to an embodiment of the present invention (A); a histogram showing the expression of SIRPα on the exosome surface by flow cytometry (B); and an electron micrograph showing an image of SIRPα-exosome (C).

FIG. 3 shows a graph showing a dynamic light scattering (DLS) analysis of the size of SIRPα-exosomes prepared according to an embodiment of the present invention (A); a transmission electron microscope image of SIRPα-exosomes (B); a histogram representing the expression of SIRPα on the exosome surface by flow cytometry (C); and a graph representing a result of a dynamic light scattering (DLS) analysis of the size of control-exosomes (D).

FIG. 4 shows a Western blot photograph showing the expression of purified recombinant SIRPα-Myc monomer protein (mSIRPα) according to an embodiment of the present invention (A); and a graph quantifying the amount of SIRPα of recombinant SIRPα-exosomes (B).

FIG. 5 is a series of graphs showing the expression of CD47 on the surfaces of HT29 (A), Raji (B) and CT26.CL25 cells (C), respectively, through cell binding assay according to an embodiment of the present invention.

FIG. 6 shows a graph showing the fluorescence intensity of HT29 cells analyzed by CD47 binding activity of recombinant SIRPα according to an embodiment of the present invention (A); a histogram showing flow cytometry analysis (B); and a graph representing mean fluorescent intensity detected in Raji and CT26.CL25 cells, respectively (C).

FIG. 7 is a series of fluorescence micrographs showing the effective binding of recombinant SIRPα in HT29 cells according to one embodiment of the present invention.

FIG. 8A shows a histogram representing a FACS analysis of the differentiation of bone marrow-derived macrophages (BMDMs) according to an embodiment of the present invention; and FIGS. 8B and 8C show a series of histograms representing FACS data obtained by analyzing the phagocytosis of cells.

FIG. 9 shows a graph representing an increase in phagocytosis by SIRPα-exosomes according to an embodiment of the present invention (A); a graph representing analysis of phagocytosis of Raji and CT26.CL25 cells by bone marrow-derived macrophages (BMDMs) (B); a graph showing a phagocytosis index (PI) (C) and a series of fluorescence microscopic images representing the appearance of HT29 cells engulfed by bone marrow derived macrophages (BMDMs).

FIG. 10 shows a graph showing tumor growth in tumor-bearing immunodeficient mice administered with SIRPα-exosomes according to an embodiment of the present invention (A); a graph showing weight of excised tumors of the tumor-bearing immunodeficient mice (B) and a series of photographs showing the excised tumors (C).

FIG. 11 represents an analysis of the antitumor effect of SIRPα-exosomes according to an embodiment of the present invention: (A) a series of photographs showing the biodistribution of Cy5.5-labeled exosomes in HT29 tumor-bearing mice; (B) a photograph showing excised tumors at 24 hours after injection of Cy5.5-labeled exosomes; and (C) a graph representing the change in the average radiation efficiency in the HT29 tumor-bearing mice.

FIG. 12 is an IVIS image obtained by injecting Cy5.5-labeled exosomes according to an embodiment of the present invention into mice showing the results of detecting fluorescence in various organs.

FIG. 13 represents the anti-tumor effect of SIRPα-exosomes according to an embodiment of the present invention using immuno-deficient and immunocompetent mice: (A) a graph showing an analysis of tumor growth inhibition in HT29 tumor-bearing immunodeficient BALB/c mice administered with SIRPα-exosome; (B) a graph showing the weight of excised tumors extracted from the immuno-deficient mice; (C) a graph showing an analysis of tumor growth inhibition in CT26.CL25 tumor-bearing immunocompetent BALB/c mice administered with SIRPα-exosome; and a graph showing the weight of the excised tumors from the CT26.CL25 tumor-bearing mice.

FIG. 14 represents an analysis of the antitumor effect of SIRPα-exosomes according to one embodiment of the present invention: (A) a series of photographs showing tumors excised from the HT29 mouse model; and (B) a series of photographs showing tumors excised from the CT26.CL25 tumor-bearing mouse model.

FIG. 15 represents an analysis of the antitumor effect of SIRPα-exosomes according to one embodiment of the present invention: (A) a graph showing the degree of binding monomeric SIRPα proteins to CD47; (B) a graph showing the phagocytic activity mediated by monomeric SIRPα in HT29 cells; and (C) a graph showing phagocytic activities mediated by monomeric SIRPα in Raji and CT26.CL25 cells, respectively.

### DETAILED DESCRIPTION

As used herein, the term “exosome” refers to a small body of lipid bilayer secreted by cells in a living organism that perform specialized functions such as coagulation, intercellular signaling and cell waste management. Alternatively, the exosome may be referred to as “extracellular vesicle” or “cell-derived membrane vesicle”. It is known to have specific nucleic acids and proteins therein and is releases into various body fluids.

As used herein, the term “SIRP (signal-regulatory protein)” refers to a regulatory membrane glycoprotein that is expressed predominantly in bone marrow cells and expressed in stem cells or neurons. Four kinds of SIRPs, i.e., SIRPα, SIRPβ, SIRPγ and SIRP8 are reported up to date. Among them SIRPα and SIRPγ are known to be inhibitory receptors and interact with the CD47 protein which is a transmembrane protein widely expressed in various cancer cells. The interaction between SIRP and CD47 is called the “don't eat me” signal. This interaction negatively regulates the effector function of innate immune cells, such as phagocytosis of tumor cells by them. This is similar to the self-signal provided by MHC I family molecules via Ig-like or Ly49 receptors. Cancer cells overexpressing CD47 activate SIRPα or SIRPγ to inhibit macrophage-mediated destruction. Recent studies have shown that high-affinity mutants of SIRPα increase the phagocytosis of cancer cells by masking CD47 on cancer cells (Weiskopf et al., Science 341 (6141): 88-91, 2013).

As used herein, the term “receptor tyrosine kinase (RTK)” refers to an important protein group that is involved in cell proliferation, differentiation, carcinogenesis, morphogenesis and the like, and the RTK includes, for example, epithelial growth factor receptor, nerve growth factor receptor, insulin receptor and hematopoietic stem cell proliferation factor receptor. The receptor activates the intracellular tyrosine kinase and transfer signals into the cell only when it binds extracellularly with these growth factors.

As used herein, the term “CD-47 binding domain” refers to a N-terminal domain that is capable of binding to CD47 and the N-terminal domain comprises up to 112 a.a.

In an aspect of the present invention, there is provided a recombinant exosome in which a phagocytosis promoting protein is presented on the surface thereof.

According to the recombinant exosome, the phagocytosis promoting protein may be a fusion protein linked to the N-terminal of the transmembrane domain of the receptor tyrosine kinase, and the receptor tyrosine kinase may be a platelet-derived growth factor receptor (PDGFR), an epidermal growth factor receptor (FGFR), vascular endothelial growth factor receptor (VEGFR), hepatocyte growth factor receptor (HGFR), tropomyosin receptor kinase (IRK), insulin receptor (IR), leukocyte receptor tyrosine kinase, angiopoietin receptor, receptor tyrosine kinase-like orphan receptors (DOR), discoidin domain receptor (DDR), rearranged during transfection receptor (RETR), tyrosine-protein kinase-like (PTK) to receptor tyrosine kinase, or MuSK (muscle-specific kinase).

Exosome is a kind of natural substance produced by cells, which is a biocompatible substance and can minimize the immune reaction. An “extracellular vesicle” or a “cell-derived membrane vesicle” included in the concept of exosome. Cell membrane protein expressed on the cell surface such as receptor can be presented on the surface by orienting it in the same direction as the cell. Thus, it is a substance that has a great advantage in presenting a cell surface protein on a surface thereof.

According to the recombinant exosome, the phagocytosis promoting protein may be SIRP or a fragment containing the CD47 binding domain of the SIRP, a Surfactant protein A, a Surfactant protein D or an anti-CD47 antibody, and the SIRP may be SIRPα, SIRPγ or high affinity variants thereof.

According to the recombinant exosome, the SIRP may comprise an amino acid sequence of any one of SEQ ID NOs: 1 to 61. According to the recombinant exosome may further include an anticancer agent. The anticancer agent may be an anti-cancer protein or an anti-cancer compound. In particular, the above-mentioned phagocytosis-promoting protein is preferably an anti-CD47 antibody or a SIRP protein which can block the signal transduction due to clustering of CD47 protein. In particular, the SIRP presented on the surface of the exosomes is clustering in the form of a lipid raft on the surface of the exosome due to the transmembrane domain of the platelet-derived growth factor receptor (PDGFR) and thus has high binding avidity to CD47 protein, which is also clustering in the form of lipid rafts on the surface of cancer cells, thereby blocks the SIRP-CD47 interaction with a small amount of exosomes, and stimulates the anti-cancer immune response more efficiently. The synergistic action of SIRPα loaded on the above exosome was first identified by the present inventors.

In the recombinant exosome, the anti-cancer protein may be an asparaginase, a protein toxin, an antibody specific for a cancer antigen, a fragment of the antibody, a tumor suppressor protein or an antiangiogenic factor. The protein toxin may be selected from the group consisting of Botulinum toxin, Tetanus toxin, Shiga toxin, Diphtheria toxin (DT), ricin, Pseudomonas exotoxin (PE), Cytolysin A (ClyA), and γ-Gelonin. The tumor suppressor protein is a protein that inhibits the development of tumors. Examples of the tumor suppressor protein include VHL (von Hippel Lindau), APC (Adenomatous polyposis coli), CD95 (cluster of differentiation 95), ST5 (Suppression of tumorigenicity 5), YPEL3 (Yippee like 3), ST7 (Supression of tumorigenicity 7), and ST14 (Suppression of tumorigenicity 14).

According to the recombinant exosome, the anticancer compound is selected from the group consisting of methotrexate, pyrimidine analogs, hydroxyurea, purine analogs, alkylating agents, immunogenic cell death inducing agents, mitotic inhibitors, angiogenesis inhibitors, intercalating agents or radionuclides.

As the anticancer compound, the following compounds can be used:
(i) methotrexate;
(ii) pyrimidine analogs
   5-fluorouracil, gemcitabine and arabinosylcytosine;
(iii) hydroxy urea;
(iv) purine analogs
   mercaptopurine and thioguanine;
(v) alkylating agents
   nitrogen mustard and cyclosporamide;
(vi) antibiotics
   anthracycline, doxorubicin, daunorubicin, idarubicin and actinomycin D;
(vii) mitotic inhibitors
   vincristine and taxol;
(viii) anti-angiogenesis agent
   anti-VEGF antibody, combretastatin A4, Fumagillin, herbimycin A, 2-methoxyestradiol, OGT 2115, TNP 470, tranilast, XRP44X, thalidomide, endostatin, salmosin, angiostatin and kringle domains of plasminogen or apolipoprotein;
(ix) intercalating agents
   carboplatin and cisplatin; and
(x) radionuclides
   ¹⁸F, ⁹⁰Y, ¹⁸⁸Re, ³²P, ⁸⁹Sr, ¹⁶⁵Dy, ¹⁸⁶Re, ¹⁹⁸Au, ¹⁵³Sm, ¹³¹I, ¹⁶⁹Er, ¹²⁵I, ⁹⁹Tc and ¹⁶⁶Ho, etc.

According to the recombinant exosome, the immunogenic cell death inducer may be selected from the group consisting of an anthracycline-type anticancer agent, an anti-EGFR antibody, a BK channel agonist, bortezomib, a cardiac glycoside+non-immunogenic cell death inducer, cyclophosphamides, a GADD34/PP1 inhibitor+mitomycin, LV-tSMAC, Measles viruse, and oxaliplatin. According to the recombinant exosome, the antracycline-type anticancer agent may be daunorubicin, doxorubicin, epirubicin, idarubicin, pixantrone, sabarubicin, or valrubicin.

In another aspect of the present invention, there is provided a pharmaceutical composition for treating cancer in a subject comprising a therapeutically effective amount of the recombinant exosome and a pharmaceutically acceptable carrier.

According to the pharmaceutical composition, one or more anticancer agents may be further included. The composition containing a pharmaceutically acceptable carrier may be various oral or parenteral formulations, but it is preferably a formulation for parenteral administration. For the preparation of formulations, a diluent or excipient such as a filler, an extender, a binder, a humectant, a disintegrant, a surfactant, etc., may be used. Solid formulations for oral administration may include tablets, pills, powders, granules, capsules, etc., and these solid formulations may be prepared by adding at least one excipient, e.g., starch, calcium carbonate, sucrose or lactose, gelatin, etc. Additionally, lubricants, such as magnesium stearate, talc, etc., may be used, in addition to the simple excipient. Liquid formulations for oral administration may include suspensions, liquid medicines for internal use, emulsions, syrups, etc., and various excipients such as humectants, sweeteners, fragrances, and preservatives, may be used, in addition to the frequently-used simple diluents such as water and liquid paraffin. Formulations for parenteral administration may include sterile aqueous solutions, non-aqueous solvents, suspensions, emulsions, lyophilized formulations, suppositories, etc. Examples of the non-aqueous solvents and suspensions may include vegetable oils such as propylene glycol, polyethylene glycol, and olive oil; an injectable ester such as ethyl oleate; etc. Examples of the bases for suppositories may include Witepsol, macrogol, Tween 61, cacao butter, laurinum, glycerogelatin, etc.

The pharmaceutical composition may have one formulation selected from the group consisting of tablets, pills, powders, granules, capsules, suspensions, solutions, emulsions, syrups, sterilized aqueous solutions, non-aqueous solutions, lyophilized formulations, and suppositories.

The pharmaceutical composition of the present disclosure may be administered orally or parenterally. When administered parenterally, the pharmaceutical composition may be administered via various routes, including intravenous injection, intranasal inhalation, intramuscular administration, intraperitoneal administration, transdermal absorption, etc.

The composition of the present disclosure may be administered in a therapeutically effective amount.

As used herein, the term “therapeutically effective amount” refers to an amount sufficient for the treatment of diseases at a reasonable benefit/risk ratio applicable to a medical treatment, and the level of the effective dose may be determined based on the factors including the kind of a subject, severity of illness, age, sex, drug activity, drug sensitivity, administration time, administration route and dissolution rate, length of treatment, factors including drug(s) to be used simultaneously in combination, and other factors well-known in the medical field. The pharmaceutical composition of the present disclosure may be administered in an amount of 0.1 mg/kg to 1 g/kg, and more preferably, 1 mg/kg to 500 mg/kg. Meanwhile, the administration dose may be appropriately adjusted according to the age, sex, and health conditions of a patient.

In another aspect of the present invention, there is provided a method of treating cancer in a subject comprising administering to a subject a therapeutically effective amount of the exosome described herein.

The composition of the present disclosure may be administered as an individual therapeutic agent, in combination with other therapeutic agents for diabetes or muscular disease, or sequentially or simultaneously with a conventional therapeutic agent(s), and it may be administered once or multiple times. It is important to administer an amount to obtain the maximum effect with a minimum amount without adverse effects considering all of the factors described above, and these factors can easily be determined by one of ordinary skill in the art.

### Examples

Hereinafter, preferred embodiments of the present disclosure will be described in detail. However, the present disclosure is not limited to embodiments explained herein but may be specified in various aspects. Rather, the embodiments are provided to sufficiently transfer the concept of the present disclosure to a person skilled in the art to thorough and complete contents introduced herein.

### Example 1: Plasma DNA Construction

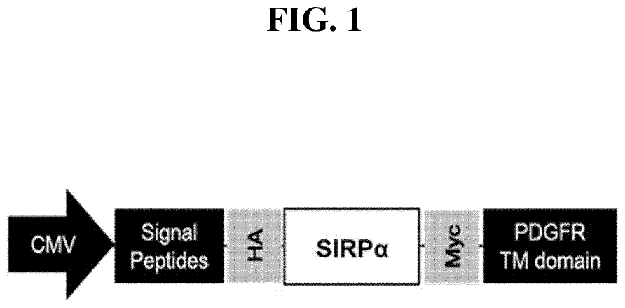

According to one embodiment of the present invention, a plasmid DNA expressing SIRPα variant (SIRPα-exosomes) which blocks CD47 on the surface of tumor cells and increases phagocytosis thereof was constructed. Specifically, the SIRPα mutant gene was obtained through a gene synthesis service (Cosmo Genetech Co.) and the DNA sequence (SEQ ID NO: 62) encoding the SIRPα variant (SEQ ID NO: 1) was inserted into frame between N-terminal signal peptide of platelet-derived growth factor receptor (PDGFR) and the membrane anchor of pDisplay vector (FIG. 1).

### Example 2: Isolation of Exosome

According to one embodiment of the present invention, HEK293T cells (6×10⁶) were cultured in Dulbecco's modified Eagle's medium (DMEM, 4,500 mg/L glucose) supplemented with 10% FBS and 1% antibiotics and the cell culture was maintained at 37° C. and 5% CO₂, and insulin-transferrin-selenium (Gibco) was added at a concentration of 80-90% confluency in a 15-cm culture dish and replaced with a serum-free DMEM. After 2 hours from the culture medium replacement, the cells were transfected with plasmid DNA (20 μg) encoding SIRPα variants using a transfection reagent (Lipofectamine 3000, Invitrogen) according to the manufacturer's instructions. After 48 hours of transfection, the cell culture supernatant was obtained by differential centrifugation to separate the exosomes, and the detailed procedure was as follows:

First, in order to remove cell debris and other cellular components in the culture medium containing exosomes, centrifugation was sequentially performed at 300 xg for 10 minutes, 2,000 xg for 10 minutes, and 10,000 xg for 30 minutes, and the culture was filtered with a 0.22 μm filter and an ultra-centrifugation was carried out at 36,900 rpm for 2 hours using a 70 Ti rotor (Beckman Instruments). The resulting recombinant exosomes (SIRPα-exosomes) were resuspended in PBS containing a protease inhibitor (Roche) and the protein concentration of the isolated exosome was measured using a BCA protein assay kit (Bio-Rad), respectively.

### Example 3: Characterization of Recombinant Exosome

The quality and characteristics of the recombinant exosome (SIRPα-exosomes) prepared according to one embodiment of the present invention are determined by western blotting (WB), flow cytometry, dynamic light scattering (DLS) and transmission electron microscope (TEM).

First, for Western blot analysis, the ultra-centrifuged recombinant exosomal pellet was dissolved using RIPA buffer (Cell Signaling Technology) containing a protease inhibitor cocktail (Calbiochem), and equivalent amount of exosomal protein (10 μg) was analyzed by SDS-PAGE and transferred to nitrocellulose membranes. Then, anti-Myc antibody (1:3000, Abcam, ab9106) and anti-HA antibody (1:500, Santa Cruz, sc-805) were added to BLAD to detect SIRPα expression, (1:500, Santa Cruz, sc-99010), anti-Tsg101 antibody (1:500, Santa Cruz, sc-22774) and anti-CD63 antibody (1:500, Santa Cruz, sc-15363) was used as exosome markers. HRP-conjugated secondary antibody (1:4,000, Sigma-Aldrich) was then added to the membrane and visualized by chemiluminescence. In addition, the expression of SIRPα on the surface of exosomes was analyzed by flow cytometry. First, 10 μg of exosomes were inserted into 4 μl aldehyde/sulfate latex beads (Invitrogen) in a final volume of 1 ml PBS for 2 hours at room temperature, then washed twice with PBS supplemented with 0.5% BSA, and the SIRPα was stained by incubating with Alexa fluor 488-conjugated secondary antibody (1:800, Jackson ImmunoResearch) and anti-Myc antibody (1:400, Abcam, ab9106) for 1 hour at 4° C. Fluorescence signals were analyzed using Accuri™ C6 flow cytometry analyzer (BD biosciences) and FlowJo_V10 software (FlowJo).

In addition, the shape of the recombinant exosome was analyzed as follows: Firstly, the recombinant exosome was located on copper grids equipped with a carbon film (Electron microscopy science), and negatively stained with uranyl acetic acid solution. The presentation of SIRPα on surface of the exosome was confirmed by immunoelectron microscopy.

Specifically, the surface SIRPα was captured using anti-Myc antibody (1:100, Abcam, ab9106) and gold-conjugated antibody (1:50, Aurion) and image was obtained using a transmission electron microscopy (Tecnai). Finally, the size distribution of the recombinant exosomes was analyzed by dynamic light scattering (DLS) using a Zetasizer Nano ZS (Malvern Instruments, Ltd., UK) and the exosome size was measured at 25° C.(Z-average) at a fixed angle of 173°.

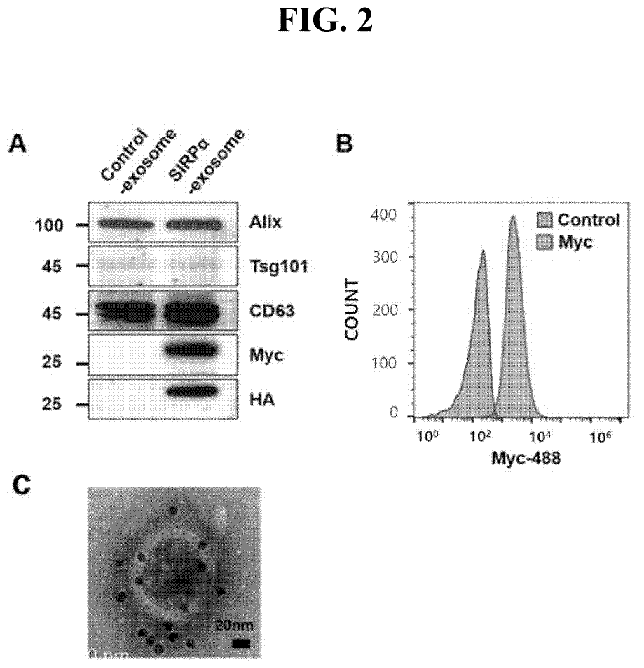

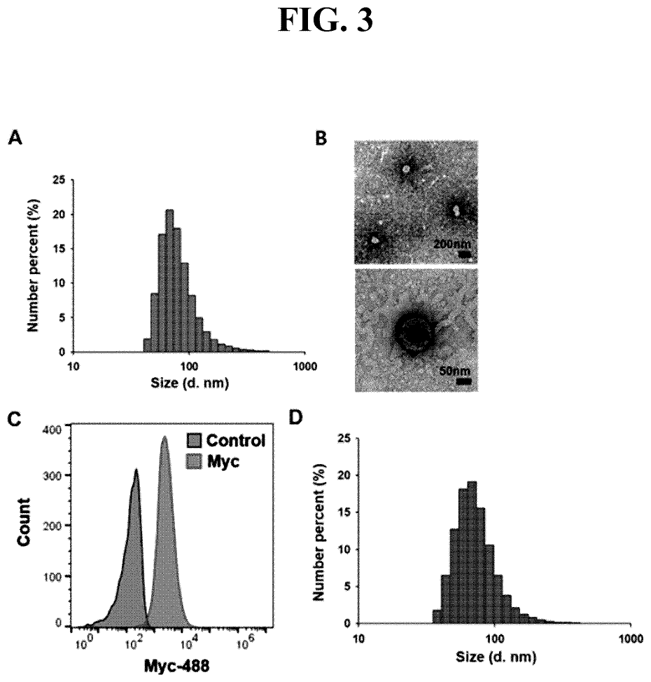

As a result, it was observed that the purified SIRPα-exosome had a SIRPα variant on the membrane thereof and contained exosomal marker proteins (FIG. 2). Also, transmission electron microscope (TEM) images and dynamic light scattering (DLS) analysis confirmed that the average size of the recombinant exosomes was 100 nm with a round shape (FIG. 3).

### Example 4: Expression and Purification of Monomeric SIRPα Protein

In order to obtain a monomeric SIPRa protein (mSIRPα) according to an embodiment of the present invention, a gene clone was obtained by PCR amplification using a primer encoding NH2-Nde I-SIRPα variant-Myc-Hind III—COOH) and the gene clone was ligated with a pET-28a plasmid vector to express SIRPα with an N-terminal histidine tag.

Subsequently, the transformed bacterial cells were cultured in LB medium containing kanamycin at 37° ° C. until the OD600 reached 0.5, and 0.5 mM isopropyl beta-D-1-thiogalactopyranoside (IPTG) (Bioneer, Korea) was added to induce protein expression. After incubation at 20° C. for 18 hours, the cells were centrifuged at 6,000×g for 10 minutes, homogenized using an ultrasonic grinder, and the pellet was resuspended in lysis buffer (1 M Tris-HCl pH 8.0, 150 mM NaCl) and mSIRPα was purified through Ni-NTA affinity column chromatography, and anti-Myc antibody (1:5,000, Abcam, ab9106) and HRP-conjugated secondary antibody (1:4,000, Sigma Aldrich) were used for western blot analysis.

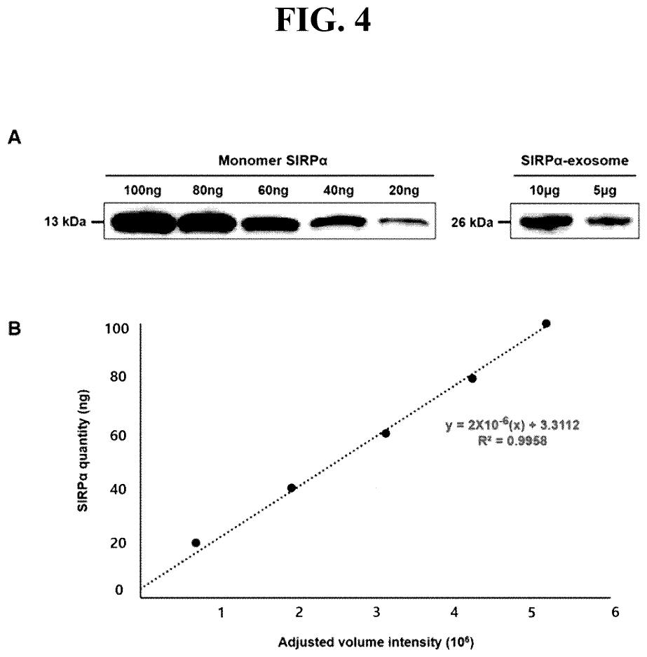

As a result, 100 μg of total protein and 2×10⁹ SIRPα-exosomes were obtained from HEK293T cells, and the amount of SIRPα was quantified using a standard curve form the western blot image of the purified recombinant SIRPα-Myc monomer protein (mSIRPα). The quantified amount of SIRPα-exosome was less than 5 ng per 1 μg of exosomes (FIG. 4). The information on the primers used in the PCR amplification is summarized in the below Table 1.

TABLE 1
Primers for the PCR amplification

| | | SEQ |
|---|---|---|
| Primer | Nucleotide sequence (5′→3′) | ID NO |
| Forward | AAA CATATG GAA GAG GAG CTG CAG | 67 |
| Reverse | AAA AAGCTT TCA ATT CAG ATC CTC TTC | 68 |

### Example 5: Cell Binding Assay

Cell binding analysis according to one embodiment of the present invention was carried out by culturing HT29 human colon adenocarcinoma (ATCC), Raji human B cell lymphoma (ATCC) and CT26.CL25 mouse colon cancer cells (ATCC) in RPMI-1640 culture medium supplemented with 10% FBS and 1% antibiotics and the cultures were maintained at 37° C. and 5% CO₂. HT29 and Raji cells were then cultured with the addition of an anti-human CD47 antibody (B6H12.2, Abcam, ab3283) and CT26.CL25 cells were incubated with anti-mouse CD47 antibody (Santa Cruz, sc-12731). For cell binding assays, HT29, Raji and CT26.CL25 cells (1×10⁶) were incubated at 4° C. for 30 minutes with the addition of PBS, exosomes or mSIRPα, respectively. The cells were then incubated with anti-Myc antibodies (1:400, Abcam, ab9106) and detected by the addition of Alexa fluor 488-conjugated secondary antibody (1:800, Jackson ImmunoResearch). The cells were then measured using an Accuri™ C6 flow cytometer (BD Biosciences) and analyzed using FlowJo_V10 software (FlowJo). The binding specificity of SIRPα-exosomes to CD47 was analyzed by block experiments through pre-incubation of cells with anti-human CD47 antibody (1:100, Abcam, ab3283).

In addition, for fluorescence microscopy analysis, HT29 cells (2×10⁵) were seeded in glass-bottom 4-well chambers and incubated with anti-Myc antibodies (1:400, Abcam, ab9106) and Alexa fluor r 488-conjugated secondary antibody (1:800, Jackson ImmunoResearch). After removal of residual non-specific signals, the cells were fixed with Hoechst 33258 for 10 min at 25° C. for 10 min followed by fixing with 4% paraformaldehyde for 7 min after nuclear staining. The cell binding capacity of exosomes was examined by fluorescence microscopy (Nikon Eclipse Ti, Nikon) and analyzed using LAS AF Lite software (Leica).

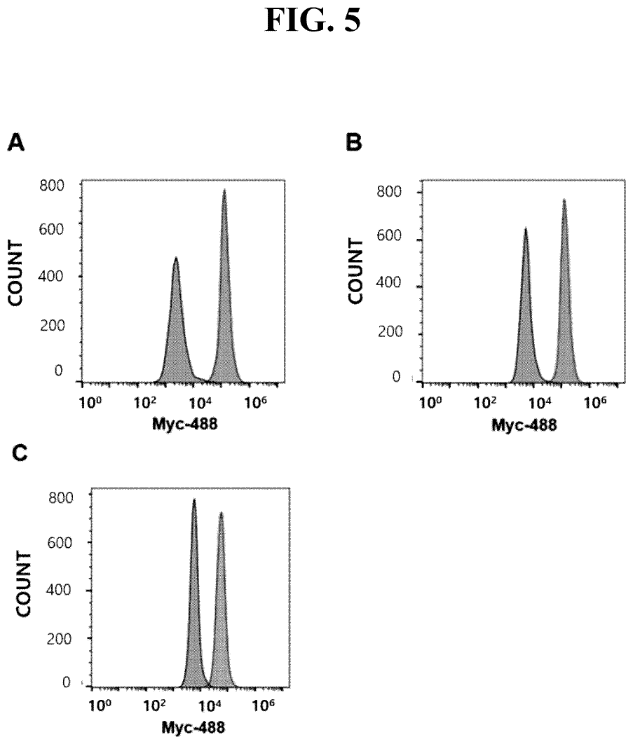

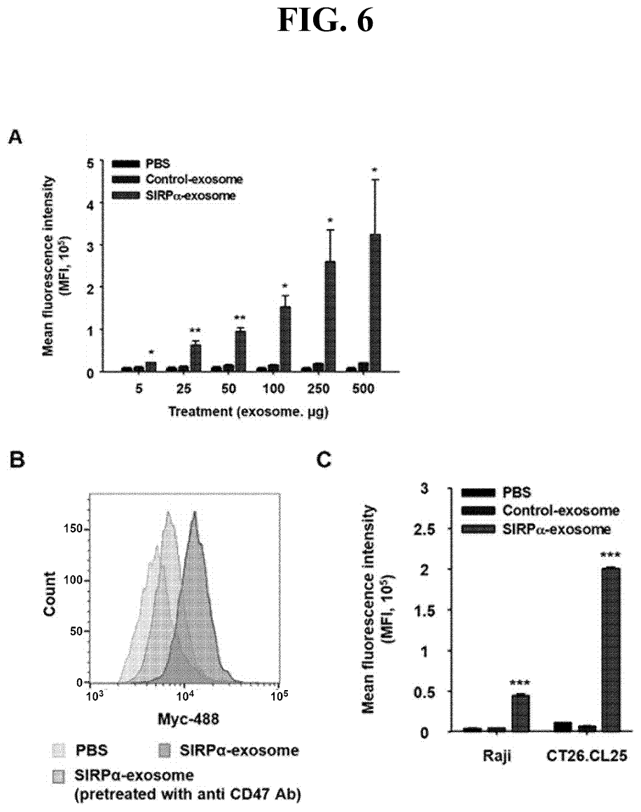

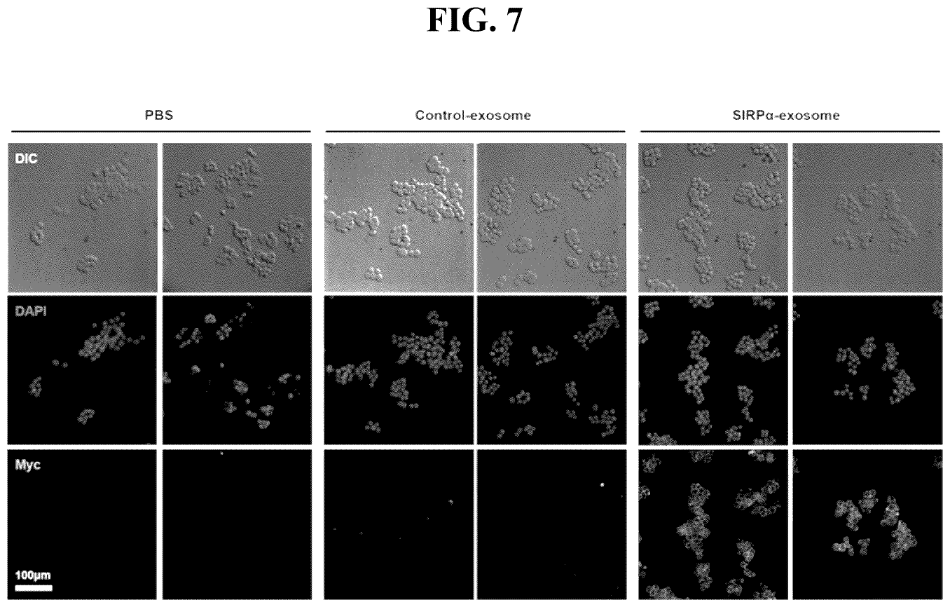

As a result, tumor cells were cultured with exosomes to confirm the ability of SIRPα-exosomes to antagonize cell surface CD47 in CD47⁺ human tumor cell lines (FIG. 5). In addition, SIRPα-exosomes showed higher affinity for CD47 in the binding of HT29 tumor cell surface compared to the exosomes of the control group whose effect was dependent on the concentration of the SIRPα-exosome. The binding of SIRPα-exosomes was reduced by preincubation with anti-human CD47 antibody, indicating that SIRPα-exosomes specifically bind to CD47 on the tumor cell surface. As well, similar results were observed in human Raji Burkitt's lymphoma (Raji) and mouse CT26.CL25 colon cancer cells for binding of SIRPα-exosomes to CD47. These results indicate that the expression of membrane-associated SIRPα variants in exosomes has the ability to bind to CD47 in cancer cells (FIGS. 6 and 7).

### Example 6: Analysis of Phagocytosis

According to one embodiment of the present invention, analysis of phagocytosis was performed to observe whether masking of CD47 with antagonistic activity of tumor cells increases phagocytosis of tumor cells. Specifically, BALB/c mice were sacrificed, and bone marrow cells were separated from leg bones to produce bone marrow-derived macrophages (BMDMs) for in vitro phagocytosis analysis. The isolated BMDMs were maintained in RPMI medium supplemented with 10% FBS and 1% antibiotic and were differentiated with macrophage colony-stimulating factor (M-CSF) for 7 days. The phagocytosis was analyzed by co-culture of BMDMs and cancer cells in serum-free RPMI medium for 4 hours. For flow cytometry, differentiated macrophages (2.5×10⁵) were stained with 0.5 μM CellTracker™ Green and the exosomes or mSIRPα proteins were preincubated with cancer cells and BMDMs were then incubated with the mixture for 4 h. The rate of phagocytosis was assessed as a percentage of double positive signals using the Accuri™ C6 flow cytometry analyzer (BD biosciences) and FlowJo_V10 software (FlowJo).

For fluorescence microscopy analysis to measure the phagocytosis index (PI), BMDMs stained with CellTracker™ Green (Thermo fisher scientific) was inoculated on a 35 mm glass-bottom dish at a density of 2.5×10⁵, and a mixture of HT29 cells (1×10⁶) was stained with pHrodo Deep red (Thermo fisher scientific) and the exosomes were treated with macrophages. After 4 hours of co-culture, the engulfment of HT29 cells by macrophages was analyzed by fluorescence microscopy (Nikon Eclipse Ti, Nikon) as a red positive signal associated with phagocytic cell formation in macrophages.

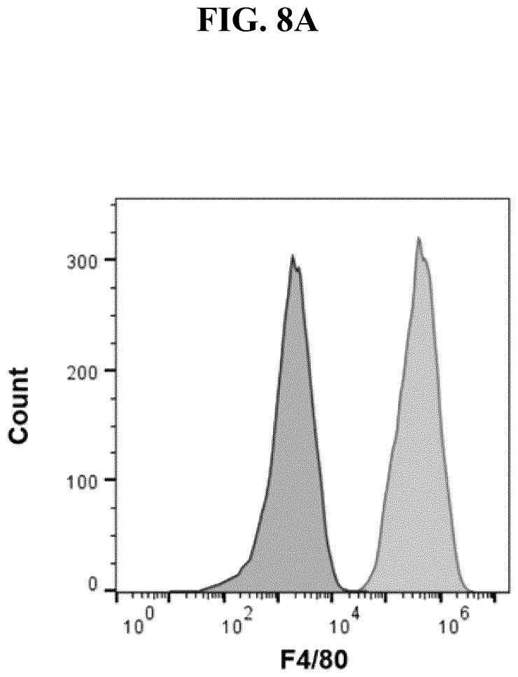

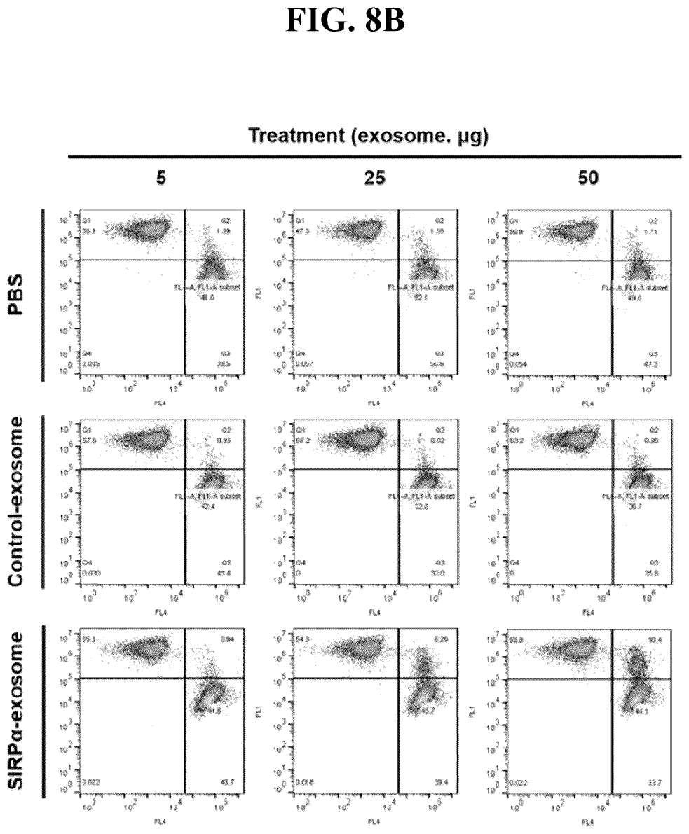

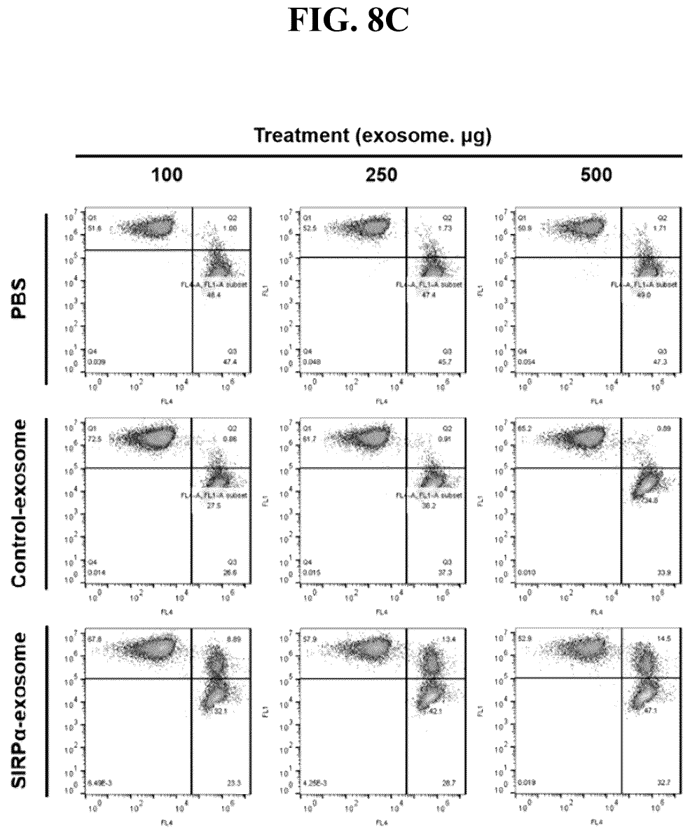

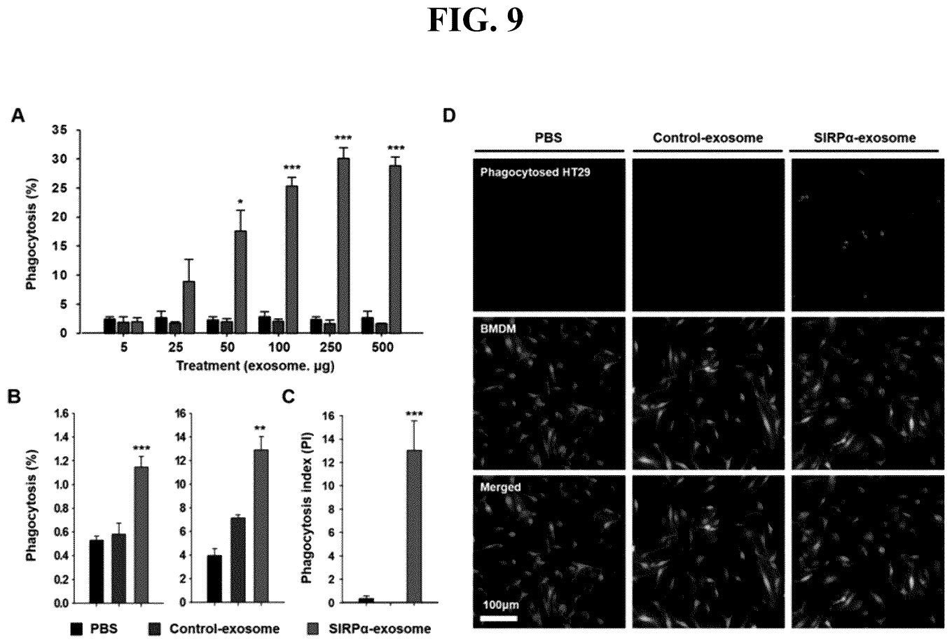

As a result, the phagocytosis by macrophages with double positive signals (deep red and green) was found to increase in a concentration-dependent manner in the SIRPα-exosome treated group compared to the control group (PBS-treated or control-exosome-treated) (FIGS. 8A-8C) and the result was similar in Raji and CT26.CL25 colon cancer cells treated with SIRPα-exosomes. In addition, treatment of SIRPα-exosomes increased the phagocytosis of tumor cells, indicating that PI was significantly increased in the SIRPα-exosome treated group (FIG. 9). Thus, blocking the interaction of CD47-SIRPα with SIRPα-exosomes is associated with an increase in the phagocytosis of various cancer cells by bone marrow-derived macrophages (BMDMs).

### Example 7: Study of In Vivo Distribution

To examine in vivo distribution of SIRPα-exosomes according to one embodiment of the present invention, exosomes were labeled with Cy5.5-NHS. After the treatment of Cy5.5-NHS dye (1 μg) to 100 μg of exosomes, the exosomes were incubated at room temperature for 2 hours and centrifuged for 45 minutes using an airfase centrifuge (Beckman coulter). The washed exosomal pellet was then resuspended in PBS after washing twice to remove unbound dye and the fluorescence intensity was measured using a fluorescence microplate reader (Infinite M200 Pro, TECAN) and normalized. In addition, Cy5.5-labeled exosomes (500 μg), free dyes and PBS were intravenously administered to HT29 tumor-bearing BALB/c nude mice respectively and the fluorescence intensity of all samples was normalized to the same value using the data obtained with fluorescence microplate reader. In vivo whole-body imaging of mice was performed at various time points (5 minutes, 2 hours, 4 hours, 8 hours, 16 hours and 24 hours) using IVIS imaging system (Caliper Life Sciences). In addition, to analyze the fluorescence intensity of the tumor, total photons per square centimeter per steradian in the ROI were calculated using Analysis Workstation software (Advanced Research Technologies Inc.) Mice were sacrificed at the time of injection and tumors and major organs including liver, lung, spleen, kidney, and heart were excised and analyzed in the same manner as above.

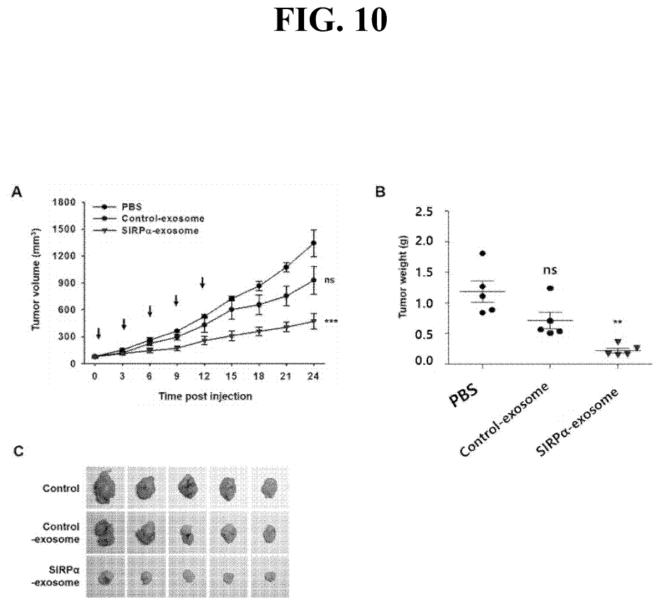

As a result, the mean weight of the excised tumors was significantly lower in the SIRPα-exosome treated group than in the control group, consistent with the observed regression of tumor growth (FIG. 10).

### Example 8: Anti-Tumor Effect Assay

Immuno-deficient BALB/c nude mice and immunocompetent BALB/c mice for in vivo experiments according to one embodiment of the present invention were used as xenograft tumor model animals at 7 weeks of age and were managed at the accommodation facilities of the Korea Institute of Science and Technology (KIST). Subsequently, HT29 cells (1×107) were subcutaneously inoculated on the left leg of BALB/c nude mice and tumors were grown for one week and then injected with control-exosomes, SIRPα-exosomes and control PBS five times every 3 days, respectively. Then, for analysis of the local anti-tumor effect, 100 μg of exosomes were injected into the tumor of the mice. For the systemic effect of SIRPα-exosomes on tumor growth, exosomes (200 μg) and PBS were intravenously injected into HT29 tumor-bearing mice 5 times every 3 days via tail vein and the tumors were excised and weighted when the tumors were grown to 1000 mm3. In addition, CT26.CL25 cells (1×10⁶) were implanted subcutaneously in the left leg of immunocompetent BALB/c mice, and after one week, 200 μg of exosomes, 200 μg of SIRPα-exosomes, 1 μg of mSIRPα (corresponding to the amount of SIRPα in 200 μg of SIRPα-exosomes), or PBS was injected through the tail vein of each of the above mice (n=7 per each group, respectively) and the tumor was excised and weighted.

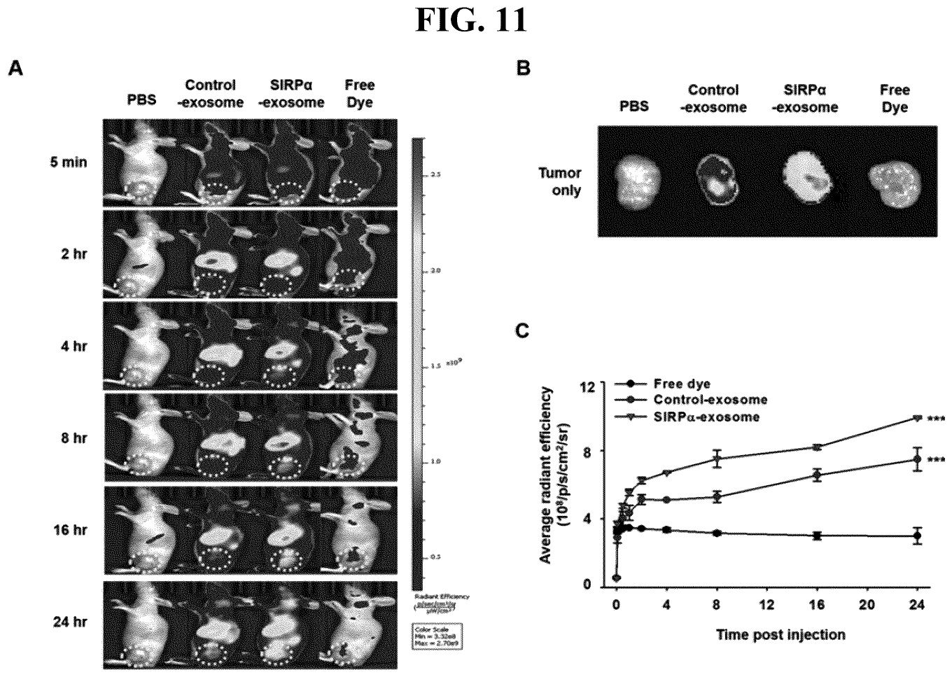

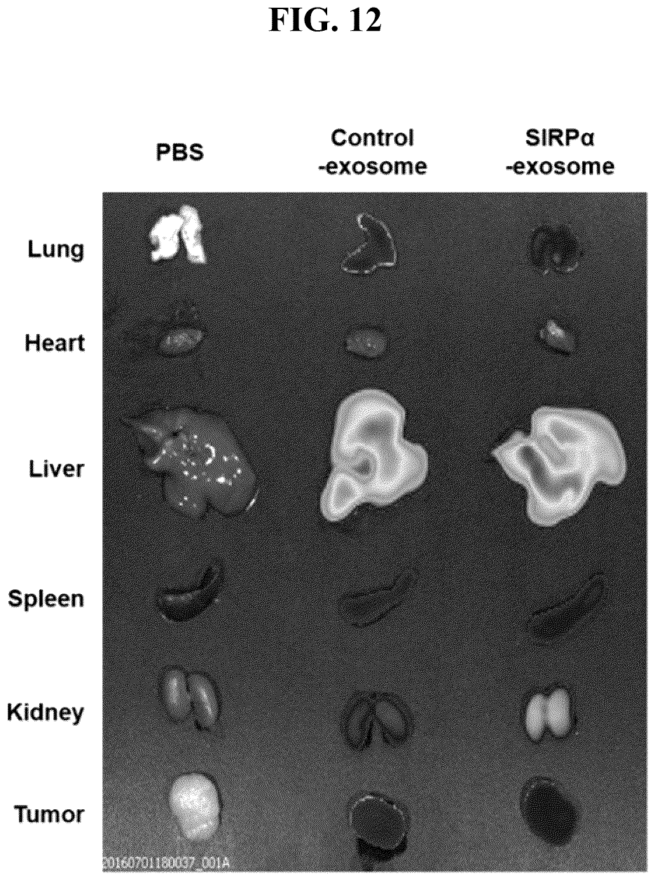

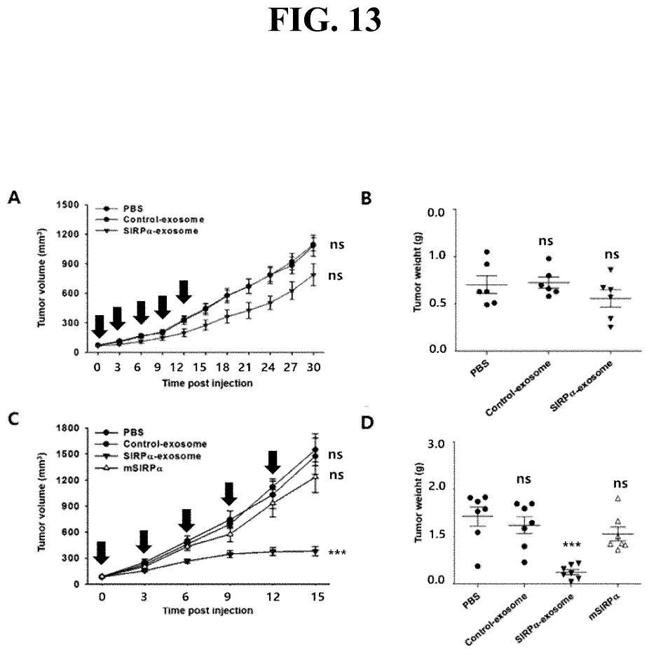

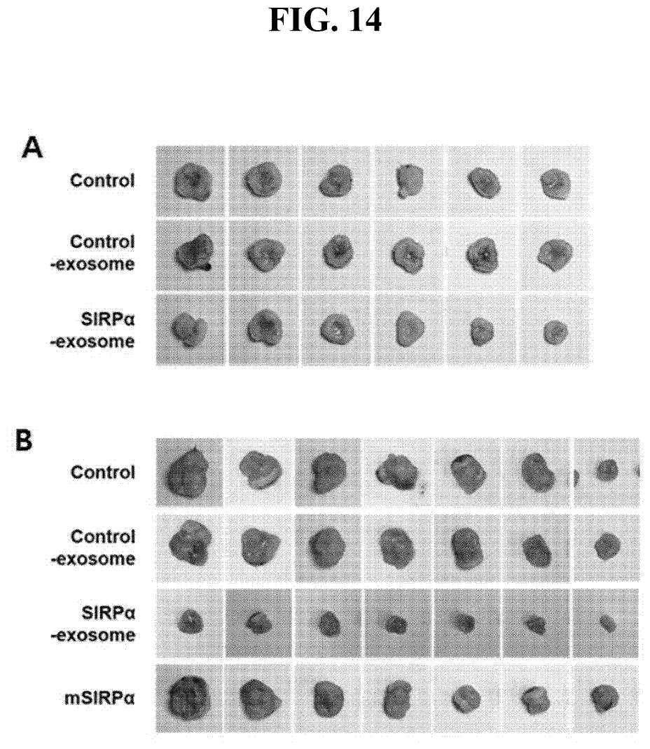

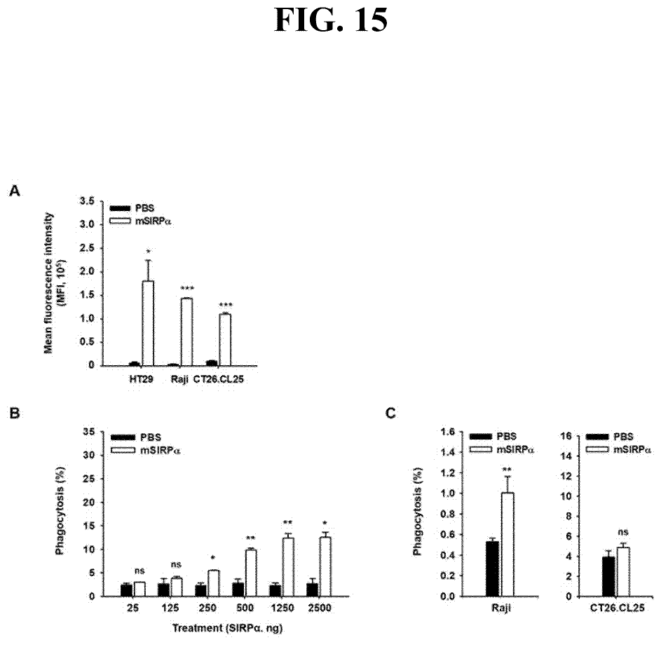

As a result, the SIRPα-exosomes labeled with Cy5.5 dye were continuously accumulated in the tumor site, indicating that not only the interaction with CD47 overexpressed in tumor cells but also the permeability and retention effect of nanoparticle-like exosomes were improved (FIG. 11). In addition, ex vivo image analysis showed that Cy5.5-labeled exosomes accumulated in the liver and kidney of the mice, supporting the results of whole animal imaging analysis (FIG. 12). Further in vivo anti-cancer effect of SIRPα-exosomes in immune-deficient mice were compared with that of the control group, but tumor growth did not show significant difference. On the other hand, anti-tumor effects of SIRPα-exosome injection were analyzed in immunocompetent mice in addition to immune-deficient mice. As a result, SIRPα-exosome slightly reduced tumor growth but was not significant, whereas the administration of SIRPα-exosomes in CT26.CL25 tumor-bearing immunocompetent BALB/c mice showed a very good anti-tumor effect. This demonstrates the remarkable anti-tumor effect of SIRPα-exosomes via immune function by administration of the SIRPα-exosomes of the present invention (FIGS. 13 and 14). On the other hand, administration of the recombinant SIRPα protein (mSIRPα) did not induce the phagocytosis of the tumor properly (FIG. 15), although it could efficiently bind CD47 of cancer cells.

In conclusion, the recombinant exosome (SIRPα-exosomes) prepared according to one embodiment of the present invention blocks the CD47-SIRPα interaction and thus increases the phagocytosis of tumor cells by macrophages and dendritic cells, and thus it can be used as a novel anticancer agent for cancer treatment.

The present disclosure has been described with reference to embodiments, but it is to be understood that they are provided herein for illustrative purposes and various changes and equivalent embodiments are possible without departing from the scope of the present disclosure by those skilled in the art. Accordingly, the true scope of protection of the present disclosure should be determined by the technical concept of the appended claims.

1. A recombinant exosome comprising on its surface a signal regulatory protein (SIRP), a fragment thereof, or a variant thereof that is fused to a protein non-naturally occurring in the exosome, wherein the protein non-naturally occurring in the exosome comprises a transmembrane domain, and wherein the recombinant exosome binds to a surface protein of a pathological cell more than a naturally occurring exosome does.

2. The recombinant exosome of claim 1, wherein the SIRP is SIRP alpha, SIRP beta, and SIRP gamma.

3. The recombinant exosome of claim 1, wherein the surface protein is CD47.

4. The recombinant exosome of claim 1, wherein the SIRP comprises the amino acid sequence of any one of SEQ ID Nos. 1-61.

5. A composition comprising the recombinant exosome of claim 1 and a pharmaceutically acceptable carrier.

6. A method of treating CD47 overexpressed pathological conditions comprising administering to a subject in need a therapeutically effective amount of the composition of claim 5.

7. The method of claim 6, wherein the SIRP is SIRP alpha, SIRP beta, and SIRP gamma.

8. The method of claim 6, wherein the surface protein is CD47.

9. The method of claim 6, further comprising administering to the subject in need a therapeutically effective amount of a therapeutic agent.

10. The method of claim 6, wherein the composition is administered orally or parenterally.

11. The method of claim 6, wherein the composition is administered intravenously, intranasally, intramuscularly, intraperitoneally, or transdermally.

12. A pharmaceutical composition comprising:
a recombinant exosome comprising on its surface a signal regulatory protein (SIRP), a fragment thereof, or a variant thereof that is fused to a protein non-naturally occurring in the exosome, wherein the recombinant exosome binds to a CD47 protein of a pathological cell;
a phagocytosis stimulating agent; and
a pharmaceutically acceptable carrier.
